(19) United States
(12) Patent Application Publication
(10) Pub. No.: US 2003/0170225 A1
(43) Pub. Date: Sep. 11, 2003

(54) Method of enhancing the effect of collagenase on wounds

(76) Inventors: Harry Soroff, Northport, NY (US); Marcia Simon, Stony Brook, NY (US); Mark S. Wolff, Setauket, NY (US); Gabriele Hatch, E. Setauket, NY (US)

Correspondence Address: JAMES & FRANKLIN, LLP, Suite 1217, 60 East 42nd Street, New York, NY, 10165, US

(21) Appl. No.: 10/094,226

(22) Filed: Mar. 11, 2002

(51) Int. Cl. A61K 38/48; A61K 31/704

(52) U.S. Cl. 424/94.63; 514/36

(57) ABSTRACT

The debridement of wounds, and particularly burn wounds, is more effectively accomplished by starting with a first substance comprising a mixture of collagenase and a non-aqueous excipient and a second substance comprising an aqueous excipient and, preferably, a cream such as an antibiotic cream, both of which substances have a satisfactory shelf life, and treating the wound by combining those two substances and promptly applying the resulting combination to the wound. Shelf life requirements are satisfied, and the effect of applying the resulting combination to the wound promptly after the combination has been formed results in a more effective debridement than would be expected from the application of other collagenase substances.

### BACKGROUND OF THE INVENTION

Collagenase has been used for years in the debridement of burns and other lesions such as dermal ulcers, particularly bed sores. The greater the speed at which the debridement takes place the sooner will the wound heal. Thus speed of debridement is a prime desideratum.

Institutions where burns are treated, such as hospitals and in particular burn centers where burn treatment is the rule and not the exception, must maintain on the shelf adequate supplies of collagenase, so as to be ready to treat burn cases as soon as they are presented, in order both to speed healing and minimize pain. It therefore is essential that the collagenase standing on the shelf waiting to be used should have as long a shelf life as possible. However, it is well known that certain collagenase compositions which are quite satisfactory when first prepared find their potency waning significantly with time. In particular, combinations of collagenase with water or aqueous excipients, while therapeutically highly acceptable, nevertheless have a shelf life at room temperatures of two weeks or less, which makes them unsatisfactory.

It has also been proposed in the past to mix the collagenase medication with other active ingredients, and in particular antibiotics and pain relief agents, with those added ingredients performing only their normal functions.

We have discovered how to produce a resultant combination of collagenase and an aqueous excipient which has a quite effective shelf life. We start with a first substance comprising collagenase in a non-aqueous excipient and a separate second substance comprising an aqueous excipient, preferably in the form of a cream such as an antibiotic cream, and we combine those two substances to produce a mixture which, when used promptly for topical wound treatment, has an improved and surprisingly rapid treatment effect as compared with applying the collagenase to the wound in a non-aqueous excipient or an aqueous excipient alone. Both the first and second substances have a highly satisfactory shelf life. When a wound is to be treated, these two substances, typically stored separately on the shelf, are combined together and then promptly applied to the wound. The resultant debridement effect, both as to extent and rapidity, is surprisingly large when compared with utilizing collagenase with a non-aqueous excipient by itself. Moreover, and quite surprisingly, the addition to the resulting combination of a common antibiotic cream not only produces the expected antibiotic effect but also enhances the debridement effect over what would be expected with the use of collagenase in either an aqueous excipient alone or a non-aqueous excipient alone. Indeed, for some reason not known to us, these results come about not just because of the presence in the applied material of collagenase, a non-aqueous excipient and an aqueous excipient, but because of the order in which they are mixed with one another. More specifically, if the antibiotic-aqueous excipient is first mixed with collagenase and the non-aqueous excipient is subsequently added, a reduction in activity, rather than an increase in activity, is observed (see Table 1, Formulation 5 below).

### DETAILED DESCRIPTION

In accordance with the present invention, a hospital or burn center will have on its shelf, ready to use when needed, first and second separate substances. The first substance will comprise collagenase and a non-aqueous excipient. A typical non-aqueous excipient is petrolatum, but others are known to the art. It is preferable to form this first substance with at least 300 units of collagenase per gram of mixture, with about 1450 units highly preferred. Amounts in excess of 2000 units are not believed to be necessary when used in accordance with the present invention, in view of the excellent debridement effect of 1450 units, but may possibly be called for in certain situations. If lower proportions of collagenase are employed the debridement results will be correspondingly lower but still clinically effective, and proportions of collagenase greater than 2000 units would be expected to produce debridement results at least as great as, and probably greater than, 2000 units.

The second substance comprises an aqueous excipient, which may preferably also include an antibiotic with or without a pain relief agent or other adjunct. Typically for use in this invention, such antibiotic is the cream form of what is sold under the trademark “Neosporin”. It contains a petrolatum excipient, but it also contains water as one of the excipients. The precise proportions of antibiotics and excipient in Neosporin are not known—the Neosporin package does not set them forth—but those proportions are not believed to be critical. While Neosporin cream as commercially available (labelled as “Neosporin and Pain Relief Cream) is known to be quite effective in connection with the present invention, other cream-type antibiotics (aqueous excipients) as well as other types of creams such as Benadryl, an antihistamine, or Cetaphil, a hand lotion are effective in the present invention. As is said in “Comprehensive Medical Chemistry”, published by Pergamon Press, “The creams used in pharmaceutical products and in the cosmetic industry are, complex emulsion formulations usually of the oil and water type, although water in oil creams can be used.” (Section 25.2, 3.1, page 572.)

Each of these first and second substances has quite an adequate shelf life, and may be stored on the shelf for an extended period of time before using.

When a burn or other appropriate wound, for example, chronic ulcers, is to be treated, quantities of the first and second substances are obtained from the shelf and mixed together to produce a combination of the two substances, and that combination is then applied to the wound in normal fashion, where it performs both its debridement function and, when an antibiotic with or without pain relief agent is included, the expected antibiotic and pain relief if appropriate, function.

The proportions of the first and second substances which make up the combination is preferably in a volume ratio of about 1:1, but that may be varied widely without departing from the present invention.

One particularly effective way in which the two mixtures may be combined for purposes of this invention is to utilize what is sometimes termed a “double barrelled mixer”, a device having two separate longitudinally extending chambers, each with its own piston initially located at one end of its chamber and with the other ends of the chambers communicating with a single exit passage in the tip of the device. To use such a device in the present invention the first and second substances are placed into the individual chambers, the pistons are put in place and connected to a static or automatic mixing tip. The exit tip of the mixer is placed close to the wound, and the two plungers are pressed, simultaneously and continuously, through their respective chambers, thus forcing the first and second substances out of their chambers into the exit passage where they mix to form a combination of the two substances, which combination then escapes from the tip of the device onto the wound.

To demonstrate the surprising debridement effect which this procedure has, we set forth the following experimental results. For these experiments we added into each well of a six well plate an 8 ml collagenase gel prepared by mixing rat tail collagen with DMEM, 10% serum, adjusting the pH to neutrality and adding human fibroblasts. Phenol red was used as a pH indicator and is not an active ingredient. To initiate the experiment, the formulations indicated below were added to the center of the upper surface of the appropriate gel and the gels were incubated over night at 37° C. After 24 hours the formulations were removed, the gels were scanned and evaluated, using the Un-Scan-It program. In Table 1 formulations 6-8 were used for purposes of comparison. The controls (Table 1 Formulation 9 and Table 2 Formulation 4), which were collagenase-free, gave background values which were subtracted from the observed values for the other formulations. For the experiment described in Table 1 there were three controls. (1) For formulations 7 and 8 the control was petrolatum alone, (2) for formulations 1-5 the control was petrolatum mixed 1:1 with Neosporin cream, and (3) for formulation 6 the control was Neosporin cream. For the experiment described in Table 2 there were three different controls. (1) For formulation 1 the control was petrolatum mixed 1:1 with Neosporin cream, for formulation 2 the control was petrolatum mixed with Benadryl Cream, and (3) for formulation 3 the control was petrolatum mixed 1:1 with Cetaphil. For the experiment described in Table 3 there were two different controls. Although both used a 1:1 mix of petrolatum and Neosporin Cream, sample 1 employed a “double-barreled mixer” and sample 2 employed hand mixing with a spatula. Note that when not indicated mixtures were made using the “double-barreled mixer”. None of the controls caused gel disintegration, and all controls were within 4% of one another.

The more collagenase activity there was, the more of the gel was degraded and the more black background visualized. Thus collagenase activity correlates positively with the number of pixels recorded in the Un-Scan-It program. Santyl is the trademark used by Advance Biofactures Corp. of Lynbrook, N.Y. for their collagenase. The other collagenase was obtained from Worthington of New Jersey, Type 1, code CLS-1. As indicated, the substances commercially labelled “Neosporin and Pain Relief Cream”, “Benadryl Cream” and “Cetaphil” were employed. The substance proportions are by volume and relate to the initial petrolatum-collagenase substance, so that the proportion of the collagenase units in the final mixture is about ½ of the indicated collagenase units in petrolatum. For each experiment, the “Average Activity” column represents a comparison with the average pixel value obtained using the results of formulation 1 as 100%.

TABLE 1
Relative effectiveness of mixtures of
non-aqueous excipients

| | | Average activity |
|---|---|---|
| | | relative to |
| Formulation | | greatest value |
| 1. | Petrolatum with 2000 units collagenase | 1.00 (±0.26) |
| | mixed 1:1 with Neosporin + Pain | |
| | Relief Cream | |
| 2. | Petrolatum with 1450 units collagenase | 0.84 (±0.13) |
| | mixed 1:1 with Neosporin + Pain | |
| | Relief Cream | |
| 3. | Petrolatum with 602 units collagenase | 0.21 (±0.03) |
| | mixed 1:1 with Neosporin + Pain | |
| | Relief Cream | |
| 4. | Petrolatum with 301 units collagenase | 0.23 (±0.15) |
| | mixed 1:1 with Neosporin + Pain | |
| | Relief Cream | |
| 5. | Neosporin + Pain Relief Cream with | −0.03 (±0.05) |
| | 1450 units collagenase mixed 1:1 | |
| | with petrolatum | |
| 6. | Neosporin + Pain Relief Cream with | 0.16 (±0.03) |
| | 1450 units collagenase | |
| 7. | Petrolatum with 1450 units collagenase | 0.10 (±0.04) |
| 8. | Collagenase Santyl (250 units | 0.03 (±0.01) |
| | collagenase) | |
| 9. | Control (0 units collagenase) | 0.00 |

TABLE 2
Relative effectivness of different
aqueous excipients

| | | Average activity |
|---|---|---|
| | | relative to |
| Formulation | | Neosporin cream |
| 1. | Petrolatum with 1450 units | 1.00 (±0.05) |
| | collagenase mixed 1:1 with | |
| | Neosporin + Pain Relief Cream | |
| 2. | Petrolatum with 1450 units | 0.98 (±0.14) |
| | collagenase mixed 1:1 with | |
| | Benadryl Cream | |
| 3. | Petrolatum with 1450 units | 0.63 (±0.05) |
| | collagenase mixed 1:1 with Cetaphil | |
| 4. | Control (0 units collagenase) | 0.00 |

TABLE 3
Relative effectiveness of two different
mixing procedures

| Mixing Procedure | | Average Activity |
|---|---|---|
| 1. | “Double-barreled mixer” used | 1.00 (±0.01) |
| | for experiments 1 and 2 | |
| 2. | “Hand mixed” using a spatula | 0.92 (±0.12) |
| | for mixing | |
| 3. | Control (0 units collagenase) | 0.00 |

While collagenase in petrolatum produces a reasonable degree of debridement if the collagenase is present in relatively large proportions such as 1450 units per gram, and while Neosporin cream containing collagenase also produces a reasonable degree of debridement (although that combination has an unsatisfactory shelf life), a product prepared in accordance with the present invention shows significantly greater degrees of debridement than comparable combinations differently prepared. Formulation 5 of Table 1 shows that when the Neosporin cream is mixed with collagenase to form a first substance, which is then mixed with petrolatum as a second substance, the treatment effect is significantly less than when the final substance is prepared in accordance with the present invention.

The use of the double-barreled mixer is by no means necessary to obtain the benefits of the present invention. See Table 3. Spatula mixing is slightly less effective than double-barreled-mixer mixing, but the latter produces more consistent mixtures and is easier to use at bedside or in the operating room.

The means used for the carrying out of the instant invention may be merchandised as a kit comprising, in separate containers or partitioned spaces, the two primary substances involved, to wit, collagenase and a non-aqueous excipient in one container and a substance comprising an aqueous excipient in the other container, possibly together with a mixing applicator such as a double-barrelled mixer described above.

The term “promptly” as here used to characterize the length of time between mixing and application means a period of time such that no significant deterioration of debridement effect results. Thus it is preferable that the end mixture be applied to the wound as soon as possible in order to obtain maximum debridement effect.

While but a limited number of embodiments of the present invention have been here specifically disclosed, it will be apparent to those skilled in the art that variations may be made therein without departing from the spirit of the invention as defined in the following claims:

1. A method for treating a wound or other condition of the body subject to topical treatment which comprises:
(a) starting with a first substance comprising collagenase and a non-aqueous excipient and a second substance comprising an aqueous excipient;
(b) combining said first and second substances; and
(c) applying the resulting combination to the wound.

2. The method of claim 1, in which the non-aqueous excipient is petrolatum.

3. The method of claim 1, in which the second substance is in the form of a cream.

4. The method of claim 2, in which the second substance is in the form of an antibiotic cream.

5. The method of claim 1, in which the second subsance is Neosporin cream.

6. The method of claim 2, in which the second substance is Neosporin cream.

7. The method of any of claims 1-6, in which the collagenase and excipient on the one hand and the second substance on the other hand are combined in a volume ratio of about 1:1.

8. The method of any of claims 1-6, in which said collagenase is present in the first substance in proportions with the excipient of at least 300 units of collagenase per gram of first substance.

9. The method of claim 7, in which said collagenase is present in the first substance in proportions with the excipient of at least 300 units of collagenase per gram of first substance.

10. The method of any of claims 1-6, in which said collagenase is present in the first substance in proportions with the excipient of about 300-2,000 units of collagenase per gram of first substance.

11. The method of claim 7, in which said collagenase is present in the first substance in proportions of about 300-2,000 units of collagenase per gram of first substance.

12. The method of any of claims 1-6, 9 and 11, in which steps (b) and (c) are carried out substantially simultaneously and continuously in the course of applying the resulting combination to a wound.

13. The method of any of claims 1-6, 9 and 11, in which steps (b) and (c) are accomplished by placing the first substance and non-aqueous excipient on the one hand and the second substance and excipient on the other hand in separate reservoirs which communicate with a common output passage, and causing the said first and second substances to move substantially simultaneously from their respective reservoirs through the common output passage to the wound.

14. The method of any of claims 1-6, 9 and 11, in which steps (b) and (c) are accomplished by placing the first substance on the one hand and the second substance on the other hand respectively in separate reservoirs of a plunger-type applicator with a common exit passage, and moving the plunger through their respective reservoirs substantially simultaneously thereby to mix the substances in the applicator and then expel the resulting combination from the applicator to the wound.

15. In combination for use in treating a burn or other wound, a first container the contents of which comprise collagenase and a non-aqueous excipient in the absence of an aqueous excipient, and a second container the contents of which comprises an aqueous excipient.

16. The combination of claim 15, together with means for mixing the contents of the said containers and applying the mixture to the wound.

17. The combination of claim 16 in which the application means is a double-barrelled mixer.

18. The combination of any of claims 15-17 in which the substance in the second container is in the form of a cream.

19. The combination of any of claims 15-17 in which the substance in the second container is in the form of an antibiotic cream.
